(19) United States
(12) Patent Application Publication
(10) Pub. No.: US 2022/0071506 A1
(43) Pub. Date: Mar. 10, 2022

(54) TRACKING AN INTERVENTIONAL DEVICE DURING AN ULTRASOUND IMAGING PROCEDURE

(71) Applicant: KONINKLIJKE PHILIPS N.V., EINDHOVEN (NL)

(72) Inventors: Peter DIRKSEN, HILVERSUM (NL); Peter Henricus Maria TIMMERMANS, TETERINGEN (NL); Jacob Roger HAARTSEN, EINDHOVEN (NL)

(21) Appl. No.: 17/465,926

(22) Filed: Sep. 3, 2021

(30) Foreign Application Priority Data
Sep. 4, 2020 (EP) 20194654.8

(51) Int. Cl. A61B 5/06; A61B 90/00; A61B 34/20; A61B 5/00

(52) U.S. Cl. CPC A61B 5/065; A61B 90/37; A61B 34/20; A61B 2562/0247; A61B 2034/2063; A61B 2034/2051; A61B 2090/3782; A61B 5/7228

(57) ABSTRACT

A mechanism for tracking the position of an interventional device from a sensing signal, generated by a capacitive pressure sensing arrangement of the interventional device, responsive to a change in pressure applied to a capacitor of the capacitive pressure sensing arrangement. The sensing signal is processed to identify a first response, which is responsive to ultrasound wave(s) incident on the capacitive pressure sensing arrangement. The first response is then further processed using a tracking algorithm to track the position of the interventional device with respect to the ultrasound transducer emitting the ultrasound wave(s).

### FIELD OF THE INVENTION

The present invention relates to the field of ultrasound imaging, and in particular, to the tracking of an interventional device during an ultrasound imaging procedure.

### BACKGROUND OF THE INVENTION

Image-guided surgical interventions allow less invasive, less traumatic procedures. One important imaging technique used in modern surgical interventions is ultrasound. On ongoing concern is how to visualize the location and/or position of an interventional device (e.g. surgical tool, such as a catheter) within a patient.

It could be possible to rely solely upon the ultrasound images generated to identify an interventional device. However, this approach is difficult and unreliable, as close proximity with tissue or other anatomical structure can mask the precise position of an interventional device.

Another approach could be to use intraoperative x-ray for visualization of tools, which show up more clearly due to the difference in contrast between interventional devices and tissue or anatomical structures. However, this method exposes patient and clinicians to ionizing radiation and can require the injection of contrast fluid for improved visibility.

Another approach is the use of electromagnetic (EM) tracking sensors, which are typically mounted aboard an interventional device (e.g. a catheter) for tracking purposes. EM tracking uses a coil that needs to be integrated into the interventional device, which can significantly increase the size of the interventional device. Moreover, the accuracy of EM tracking can become extremely poor in an EM distorted operating environment, e.g. the presence of metal. Furthermore, an independent EM tracking system adds to the equipment cost and clutter in surgical environment.

It would therefore be preferably to provide an interventional device tracking mechanism that minimizes the need for additional equipment and does not expose a patient or clinician to potentially hazardous materials and/or environs.

U.S. Pat. No. 4,249,539 describes a biopsy needle with an active ultrasound transducer at its tip for clear identification of the needle tip. The transducer at the tip of the needle, upon sensing signals from an imaging probe, immediately transmits back a short pulse. The ultrasound transducer on the needle thus acts as a “super-reflector” that re-radiates a strong ultrasonic signal upon insonification. This facilitates ease of identifying an interventional device solely from ultrasound imaging, with improved reliability.

### SUMMARY OF THE INVENTION

The invention is defined by the claims.

According to examples in accordance with an aspect of the invention, there is provided a sensing arrangement for tracking a position of an interventional device, within a patient, with respect to an ultrasound transducer that emits ultrasound waves according to a predetermined modulation pattern, wherein the interventional device comprises a capacitive pressure sensing arrangement.

The sensing arrangement comprises: an input interface configured to obtain a sensing signal from the capacitive pressure sensing arrangement of the interventional device, wherein the sensing signal is responsive to a change in a capacitance of a capacitor of the capacitive pressure sensing arrangement that results from a change of pressure induced on the capacitor; a processing arrangement configured to: generate a measure of pressure by processing the sensing signal, wherein the measure of pressure is a measure of pressure resulting from one or more anatomical features of the patient; and identify a first response of the sensing signal, the first response being a response of the sensing signal to the ultrasound waves emitted by the ultrasound transducer, by processing the sensing signal based on the predetermined modulation pattern; and track the position of the interventional device with respect to the ultrasound transducer using the first response of the sensing signal.

The present disclosure recognizes that a signal generated by a pressure responsive capacitor, which can be used to derive a measure of pressure (e.g. fraction flow reserve) that results from one or more anatomical features of the patient, can also be used to identify a magnitude of incident ultrasound waves on the capacitor without requiring any modification to the operation of the capacitive pressure sensing arrangement itself (e.g. without biasing or the like).

In particular, it is possible to simultaneously track the position of an interventional device and monitor a measure of pressure (resulting from anatomical features), e.g. without needing to switch a mode of operation.

The present disclosure recognizes that an ultrasound wave causes an apparent pressure increase on a sensing signal (which can be otherwise processed to derive the measure of pressure). Thus, the positional relationship between the ultrasound transducer (producing the ultrasound waves) and the capacitive pressure sensing arrangement can be determined using the response of the capacitive pressure sensing arrangement to the ultrasound waves.

To facilitate discrimination between changes in the sensing signal resulting from the ultrasound waves, and changes resulting from natural variation in pressure surrounding the capacitive pressure sensing arrangement (e.g. changes in blood pressure), the ultrasound waves are emitted according to a modulation pattern to facilitate identification of changes caused by the ultrasound waves (as these changes will follow the modulation pattern). The modulation pattern may be an ON-OFF (e.g. start-stop) modulation pattern and/or an ultrasound wave intensity modulation pattern.

Preferably, the capacitive pressure sensing arrangement comprises a capacitor having one or more membranes that deflect in response to a change of pressure (induced on the capacitor), thereby resulting in a change in capacitance of the capacitor. In other words, the capacitance of the capacitor of the capacitive sensing arrangement may be responsive to a deflection of the one or more membranes of the capacitor. More broadly, the sensing signal may be responsive to a change in deflection of the one or more membranes.

The processing arrangement may be configured to generate the measure of pressure by: processing the sensing signal based on the predetermined modulation pattern to determine a second response of the sensing signal, the second response being a response of the sensing signal to a change in a capacitance of the capacitive pressure sensing arrangement that does not result from the ultrasound waves emitted by the ultrasound transducer; and generating the measure of pressure based on the second response of the sensing signal.

Thus, the processing arrangement may take account of the effect of the ultrasound wave on the sensing signal when generating the measure of pressure. In particular, a second response (being a response of the sensing signal free of the influence of the ultrasound wave(s)) can be generated and subsequently processed to generate the measure of pressure.

As one example, the second response may be generated by identifying the first response, and subtracting the first response from the sensing signal to generate the second response. This approach will generate a sensing signal free from the influence of any ultrasound waves.

Optionally, the processing arrangement is configured to track the position of the interventional device by: determining a difference between a first value of a parameter of the sensing signal and a second value of the same parameter of the sensing signal, wherein the first value is obtained whilst the sensing signal is modified by an ultrasound wave and the second value is obtained whilst the sensing signal is not modified by any ultrasound wave; and processing the determined difference using a tracking algorithm to thereby track the position of the interventional device.

The present disclosure recognizes that there is a relationship between position of the interventional device and magnitude of the perceived change in pressure of a sensing signal resulting from the ultrasound waves (caused by the ultrasound waves vibrating a membrane of the capacitor). This is because different positions will result in the interventional device being at different distances from the ultrasound transducer, meaning that the energy of the ultrasound wave incident on a more distant interventional device will be less than the energy at a more proximate interventional device.

In this way, a difference between the two values of the parameter of the sensing signal can be used to derive changes in the distance between the ultrasound transducer and the interventional device, and thereby a position of the interventional device.

Preferably, the measure of pressure is a measure of fractional flow reserve. Other suitable measure of pressures will be apparent to the skilled person, e.g. diastolic pressure, systolic pressure, ratio between systolic over diastolic and so on).

The sensing arrangement may further comprise an output interface configured to provide one or more output signals responsive to the tracked position of the interventional device and/or the measure of pressure. Preferably, each output signal is a display signal configured to control a display provided by a user interface responsive to the tracked position of the interventional device and/or the measure of pressure.

In some examples, the sensing signal provides an alternating signal, the frequency of the alternating signal being responsive to a change in a capacitance of a capacitor of the capacitive pressure sensing arrangement that results from a change of pressure induced on the capacitor.

There is also proposed a sensing system for tracking a position of an interventional device, within a patient, with respect to an ultrasound transducer that emits ultrasound waves, wherein the interventional device comprises a capacitive pressure sensing arrangement.

The sensing system comprises the sensing arrangement previously described and a control interface configured to control all signals provided to the capacitive pressure sensing arrangement and to receive all signals generated or modified by the capacitive pressure sensing arrangement, wherein the control interface is configured to provide and/or receive only two signals from or to the capacitive pressure sensing arrangement.

One key advantage of the proposed mechanism is that it can be used with capacitive pressure sensing arrangements that have only two terminals/electrodes for receiving/sending signals (including power signals and ground/reference voltage signals). Thus, a dedicated sensor line is not required for use with embodiments of the present invention.

This means that the size of the interventional device and related components can be made smaller, whilst still facilitating tracking of the interventional device.

There is also proposed an interventional medical system comprising: an interventional device comprising a capacitive pressure sensing arrangement having a capacitor configured to change capacitance responsive to a pressure induced thereon, wherein the interventional device is configured to generate the sensing signal responsive to a change in a capacitance of a capacitor of the capacitive pressure sensing arrangement that results from a change of pressure induced on the capacitor; and the sensing arrangement or the sensing system previously described.

The interventional device may comprise: a first electrode, configured to receive a first signal; and a second electrode; configured to receive a second signal, wherein the capacitive pressure sensing arrangement is configured to modify the first and/or second signal responsive to a change in the capacitance of the capacitor.

In some examples, where the interventional medical system comprises a sensing system, the control interface of the sensing system is configured to: provide, to the first electrode, a voltage for powering the capacitive pressure sensing arrangement; and provide, to the second electrode, a ground voltage. In other words, the first signal may comprise a voltage supply signal and the second signal may comprise a ground/reference voltage signal. These may be the only signals received from or passed to the interventional medical device.

As previously explained, the capacitive pressure sensing arrangement may be configured to modify the voltage for powering the capacitive pressure sensing arrangement responsive to a change in capacitance of a capacitor of the capacitive pressure sensing arrangement. This can be performed, for example, by alternately connecting and disconnecting the first electrode and the second electrode at a frequency responsive to the capacitance of the capacitor of the capacitive pressure sensing arrangement.

The interventional medical system preferably further comprises an ultrasound transducer configured to emit ultrasound waves according to a predetermined modulation pattern. In other examples, the ultrasound transducer is provided by an external device or system.

The interventional medical system may further comprise an ultrasound system configured to control the operation of the ultrasound transducer. The sensing arrangement may communicate with the ultrasound system and/or ultrasound transducer to obtain information about the predetermined modulation pattern.

There is also proposed a computer-implemented method for tracking a position of an interventional device, within a patient, with respect to an ultrasound transducer that emits ultrasound waves according to a predetermined modulation pattern, wherein the interventional device comprises a capacitive pressure sensing arrangement.

The computer-implemented method comprises: obtaining a sensing signal from the capacitive pressure sensing arrangement of the interventional device, wherein the sensing signal is responsive to a change in a capacitance of a capacitor of the capacitive pressure sensing arrangement that results from a change of pressure induced on the capacitor; generating a measure of pressure by processing the sensing signal, wherein the measure of pressure is a measure of pressure resulting from one or more anatomical features of the patient; identifying a first response of the sensing signal, the first response being a response of the sensing signal to the ultrasound waves emitted by the ultrasound transducer, by processing the sensing signal based on the predetermined modulation pattern; and tracking the position of the interventional device with respect to the ultrasound transducer using the first response of the sensing signal.

There is also proposed a computer program product comprising instructions which, when executed by a suitable computer or processing system, cause the computer to carry out the method herein described.

The present disclosure also proposes a computer program (product) comprising instructions which, when the program is executed by a computer or processing system, cause the computer or processing system to carry out (the steps of) any herein described method. The computer program (product) may be stored on a non-transitory computer readable medium.

Similarly, there is also proposed a computer-readable (storage) medium comprising instructions which, when executed by a computer or processing system, cause the computer or processing system to carry out (the steps of) any herein described method. There is also proposed computer-readable data carrier having stored thereon the computer program (product) previously described. There is also proposed a data carrier signal carrying the computer program (product) previously described.

The skilled person would be readily capable of adapting any herein described method to reflect embodiments of herein described apparatus, systems and/or processors, and vice versa. A similar understanding would be made by the skilled person with respect to a computer program (product).

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a capacitive pressure sensing arrangement for use in an embodiment;

FIG. 2 illustrates an interventional device for use in an embodiment;

FIGS. 3 and 4 illustrate a relationship between a sensing signal generated by a capacitive pressure sensing arrangement and a magnitude of ultrasound waves incident on the capacitive pressure sensing arrangement;

FIG. 5 illustrates an interventional medical system according to an embodiment;

FIG. 6 illustrates a method according to an embodiment; and

FIG. 7 illustrates an ultrasound system.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a mechanism for tracking the position of an interventional device from a sensing signal, generated by a capacitive pressure sensing arrangement of the interventional device, responsive to a change in pressure applied to a capacitor of the capacitive pressure sensing arrangement. The sensing signal is processed to identify a first response, which is responsive to ultrasound wave(s) incident on the capacitive pressure sensing arrangement. The first response is then further processed using a tracking algorithm to track the position of the interventional device with respect to the ultrasound transducer emitting the ultrasound wave(s).

The present disclosure relies upon the recognition that ultrasound waves cause (a membrane of) a capacitor of a capacitive pressure sensing arrangement to undergo vibration, particularly harmonic vibration. As the relationship between pressure applied to a capacitor and the change in height of the capacitor is non-linear, the vibrations causes a perceived pressure increase on the capacitor. This perceived pressure increase can be identified from the sensing signal, and used to determine an intensity of ultrasound waves incident on the capacitive pressure sensing arrangement, thereby allowing a location of the capacitive pressure sensing arrangement (and therefore interventional device) with respect to the ultrasound transducer emitting the ultrasound waves to be tracked and/or identified.

Embodiments of the invention can be employed in any suitable medical interventional system which employs pressure sensing, e.g. for insertion of a catheter or investigation of a lesion within a blood vessel.

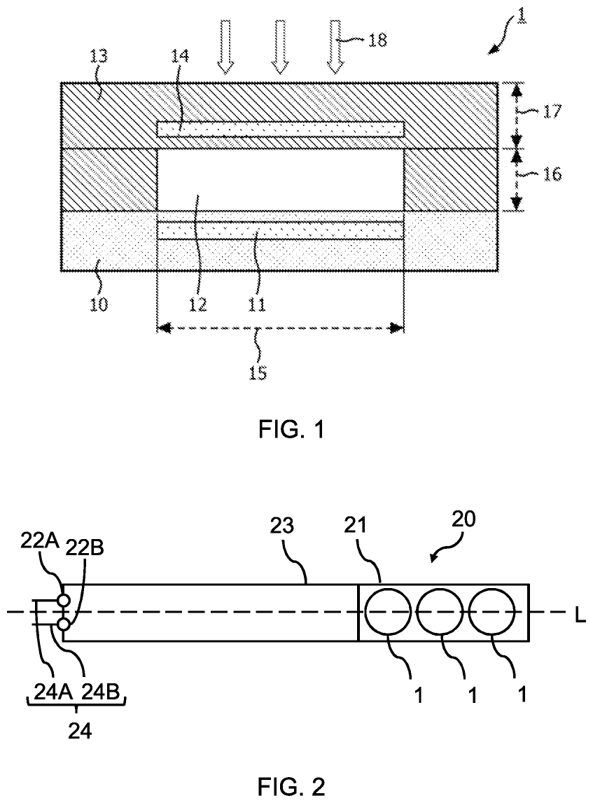

FIG. 1 illustrates a capacitive pressure sensing arrangement 1 usable in an interventional device an embodiment of the invention. The drawing has been simplified to explain the features most relevant to the understanding of the invention. If should be noted that the capacitive pressure sensing arrangement may comprise other features or layers or layer stacks, as necessary for the processing and electrical operation of the capacitive pressure sensing arrangement and/or interventional device. For example, the external connections to a possible associated ASIC or any other connections to the outside environment are not shown here.

Broadly speaking, the capacitive pressure sensing arrangement 110 comprises a capacitor with a capacitance that changes as pressure induced on the capacitor changes. The structure of the capacitor is analogous to a capacitive micromachined ultrasonic transducer (CMUT), and is a microelectromechanical system (MEMS) that responds to a change in pressure. The capacitive pressure sensing arrangement is configured to generate a sensing signal responsive to the change in capacitance of the capacitor.

As an example only, the illustrated capacitive pressure sensing arrangement, is founded on a silicon substrate 10. This silicon substrate 10 is provided with a first electrode 11, commonly known as the bottom electrode, which may be directly in contact with the silicon substrate 10 or may be arranged close to the silicon substrate 10 but separated by some other base processing layers. A cavity 12 is provided—this cavity is normally kept at low pressure close to vacuum and provides a space between the silicon substrate 10 and a silicon nitride membrane 13. The silicon nitride membrane 13 may also be biased into a so-called “collapsed” mode, in which instance the membrane may be (partly) in contact with the silicon substrate 10 due to the application of an applied voltage. The silicon nitride membrane 13 has a second electrode 14 embedded in it. This second electrode 14 forms an electrode pair with the first electrode 11 and is commonly known as the “top” electrode. The capacitive effect of the structure comes from the provision of these two electrodes 11 and 14. That is, the first electrode 11 and the second electrode 14, together with the cavity 12, effectively form a capacitor within the capacitive pressure sensing arrangement. A common length value of the electrodes 11 and 14 as indicated by arrow 15 is 25 to 250μιη diameter of a circular device. The electrodes 11 and 14 are generally manufactured to be of similar length. A typical height of the vacuum cavity 12 is around 0.5μιη, as indicated by arrow 16. A typical height of the silicon nitride membrane 13 is around 0.5μιη, as indicated by arrow 17. In operation, the silicon nitride membrane 13 experiences a pressure, as indicated by arrow 18, which causes the silicon nitride membrane 13 to flex. The change in position of the silicon nitride membrane 13 causes a change in distance between the first and second electrodes 11 and 14, thereby changing a capacitance established between them.

This change in capacitance can be detected and indicated in a sensing signal. For instance, the change in capacitance can be converted into a pressure measurement change to indicate the pressure change.

The capacitive pressure sensing arrangement may further comprise a circuit arrangement (not illustrated), e.g. on the substrate 10, adapted to generate a sensing signal based on a capacitance between the two electrodes of the capacitor; and a pair of terminals/electrodes conductively coupled to the circuit arrangement for receiving power supply wires and providing the sensing signal to an external device. The circuit arrangement may, for example, comprise an ASIC.

In one example, the circuit arrangement, may be adapted to modulate the power supply with the processed sensor signal (i.e. control a frequency of a power supply), such that the capacitive pressure sensing arrangement does not require a separate signal terminal/electrode and only needs connecting to two power supply wires. In other words, a power supply may be modified by the capacitive pressure sensing arrangement to act as a sensing signal.

Other suitable approaches for modifying power supply signals to facilitate provision of information about the capacitance of the capacitor of the capacitive pressure sensing arrangement to external devices (e.g. a provider or monitor of the power supply) will be apparent to the skilled person.

The circuit arrangement may perform some (pre-)processing of the sensing signals, such as digitization and/or amplification such that the capacitive pressure sensing arrangement itself can be kept as small as possible. Without such signal processing circuitry, the physical dimensions of the capacitive pressure sensing arrangement are limited by the length of wire(s) over which the sensor signals are to be carried to a user console or the like, as signal losses over such wires dictate that the initial signal strength of the signals produced by the capacitive pressure sensing arrangement is sufficient such that a processable residual sensor signal is received by the user console after signal losses. Consequently, the inclusion of the signal processing circuit arrangement in the substrate ensures that the sensor signals may be weaker, i.e. that the capacitive pressure sensing arrangement may be smaller.

The capacitive pressure sensing arrangement is thereby able to generate a sensing signal responsive to pressure applied to a capacitor of the capacitive pressure sensing arrangement. One or more parameters of the sensing signal (e.g. (average) magnitude, maximum amplitude, minimum amplitude or frequency) is made responsive to the pressure applied to the capacitor of the capacitive pressure sensing arrangement.

In some examples, such as where the power supply to the capacitive pressure sensing arrangement is modulated, the capacitive pressure sensing arrangement can provide an alternating signal, the frequency of the alternating signal being responsive to a change in a capacitance of a capacitor of the capacitive pressure sensing arrangement that results from a change of pressure induced on the capacitor of a capacitive pressure sensing arrangement. This can be performed by selectively coupling the power supply to a ground/reference voltage at a frequency responsive to the capacitance of the capacitor of the capacitive pressure sensing arrangement.

In particular examples, the lower the frequency of the alternating signal, the greater the pressure applied to the capacitor of the capacitive pressure sensing arrangement. Suitable circuitry for carrying out this approach will be apparent to the skilled person.

The skilled person would be readily capable of using any other suitable capacitive pressure sensing arrangement for generating a sensing signal responsive to a change in capacitance (of a capacitor of the capacitive pressure sensing arrangement) resulting from a change in pressure induced on the capacitor. As such, the capacitive pressure sensing arrangement illustrated in FIG. 1 is only a suitable example.

In particular, the invention is particularly advantageous when the capacitive pressure sensing arrangement comprises a capacitor having one or more membranes that deflect in response to a change of pressure, thereby resulting in a change in capacitance of the capacitor. This is because these such membranes are particularly susceptible to vibration in the presence of ultrasound waves, and therefore to having a change in capacitance.

Some capacitive pressure sensing arrangements may comprise a plurality of capacitors (e.g. a plurality of membranes) which the capacitance of each capacitor is responsive to a change in pressure. This capacitors may be connected in parallel or series. The proposed invention is particularly advantageous if the capacitive pressure sensing arrangement comprises more than one membrane responsive to a change in pressure, for reasons set out below.

By way of example, another suitable capacitive pressure sensing arrangement (which is effectively an example of pressure sensor) is described by International Patent Application No. WO 2019/166263 A1.

FIG. 2 schematically depicts an example interventional device 20 for insertion into a patient for use in an embodiment. The interventional device may, for example, be in the form of a catheter, a guide wire, a sheath or other invasive medical device.

The interventional device 20 comprises a portion 21 that is typically inserted into a patient during use of the interventional device 20. The portion 21 carries one or more capacitive pressure sensing arrangements 1, such as those previously described. In a preferred embodiment, the capacitive pressure sensing arrangement(s) is/are operable to monitor local blood pressure within an artery or a vein of the patient. For example, the capacitive pressure sensing arrangements may be deployed to determine a gradient in the blood pressure of the patient between a first location and a second location within the artery or vein of the patient, which may be indicative of the presence of a narrowing anomaly such as a lesion or stenosis in between the first and second locations.

The interventional device may be configured to comprise only two electrodes 22A, 22B or terminals for communicating with and receiving power from external devices. A first electrode 22A can be used to receive a power supply and a second electrode can be used to receive a ground/reference voltage. The power supply received at the first electrode may be modified (e.g. modulated) by a capacitive pressure sensing arrangement based on the capacitance of the capacitor of the capacitive pressure sensing arrangement. In this way, a sensing signal generated by a capacitive pressure sensing arrangement can be output to an external device, via the electrodes of the interventional device.

In some examples, the interventional device 20 comprises a single capacitive pressure sensing arrangement 1. The electrodes of the interventional device may thereby be directly connected to the capacitive pressure sensing arrangement to thereby output the sensing signal generated by the single capacitive pressure sensing arrangement, i.e. the capacitive pressure sensing arrangement may directly modify the power supply provided at the first and second electrodes of the interventional device 20.

Optionally, the interventional device 20 comprises a plurality of capacitive pressure sensing arrangements 1 organized in an array along the elongation direction L of the interventional device 50. The interventional device may be configured to alternately provide the sensing signals generated by different capacitive pressure sensing arrangements, e.g. using a time-division multiple access technique, to the first and second electrodes. This can be performed, for example, by switching which capacitive pressure sensing arrangements are able to draw power from the power supply provided at the first electrode 22A of the interventional device (e.g. using a suitable switching arrangement).

In such an array, the capacitive pressure sensing arrangements are typically spatially separated from each other in the elongation direction L such that the capacitive pressure sensing arrangements 1 may simultaneously determine the local blood pressure in a plurality of locations within the artery or vein of the patient with a portion 21 such that the location of any anomaly can be associated with the position of the one or more capacitive pressure sensing arrangements across which the gradient in the blood pressure is observed such that upon determination of the position of the portion 21 within the vein of artery of the patient, the location of the anomaly can be accurately pinpointed.

The interventional device 20 may further comprise a body 23 onto which the portion 21 is mounted through which wires 24 extend, e.g. bifilar wires within the body 23. As previously explained, in a preferred embodiment only two of such wires 24 are required, such that the sensor signal is added by a circuit arrangement (e.g. an ASIC) to the power supplied over these wires.

In other words, the interventional device may comprise a first electrode, configured to receive a first signal (over a first wire 24A); and a second electrode; configured to receive a second signal (over a second wire 24B), wherein the capacitive pressure sensing arrangement and/or interventional device is configured to modify the first and/or second signal responsive to a change in the capacitance of the capacitor.

The wires 24 are typically connected to the respective electrodes of the one or more capacitive pressure sensing arrangements 10 and to connectors at a distal end of the interventional device 20 relative to the portion 21 such that the one or more capacitive pressure sensing arrangements 10 can be connected to a control module or user console through which the one or more capacitive pressure sensing arrangements 10 can be controlled. As such arrangements are well-known per se, they are not explained in further detail for the sake of brevity only. It suffices to say that any suitable arrangement for connecting the one or more capacitive pressure sensing arrangements 10 to such a control module may be used. The interventional device 20 may take any suitable shape, such as a catheter or a guide wire. Due to the compact nature of the capacitive pressure sensing arrangement 10, i.e. having a height or thickness as little as 70 μm, the capacitive pressure sensing arrangement 10 according to embodiments of the present invention can be used on extremely thin invasive interventional devices, e.g. catheters that fit in a blood vessel having a diameter in a range of 0.5-10 mm. For example, the capacitive pressure sensing arrangement 10 may be used on a FFR catheter for heart applications having a typical diameter of about 0.3 mm.

For the sake of completeness, it is noted that the interventional device may comprise one or more other components, sensors or surgical tools (e.g. ablation electrodes or the like) for use in performing analysis, monitoring and/or surgical procedures on the patient. These elements have not been illustrated for the sake of clarity.

The present disclosure relates to a new mechanism for processing signals generated by an interventional device having a capacitive pressure sensing arrangement, such as those previously described. In particular, a new mechanism for enabling the tracking of a position of an interventional device having a capacitive pressure sensing arrangement is hereafter described.

The present disclosure recognizes that an ultrasound wave incident upon a capacitive pressure sensing arrangement will cause (the membrane of) a capacitor of the capacitive pressure sensing arrangement to vibrate. In particular, an ultrasound wave can cause the (membrane of the) capacitor to undergo harmonic vibration.

It has been further recognized that the change in capacitance of a capacitor of a capacitive pressure sensing arrangement is not directly proportional to a change in height applied to a membrane of a capacitor. Thus, incident ultrasound waves will result in an average capacitance of the capacitor of the capacitive pressure sensing arrangement increasing. The capacitance of a capacitive pressure sensing arrangement increases as pressure applied to the membrane of the capacitor increases, so that the incidence of ultrasound waves on a capacitor creates an apparent/perceived increase in pressure (as indicated in the sensing signal).

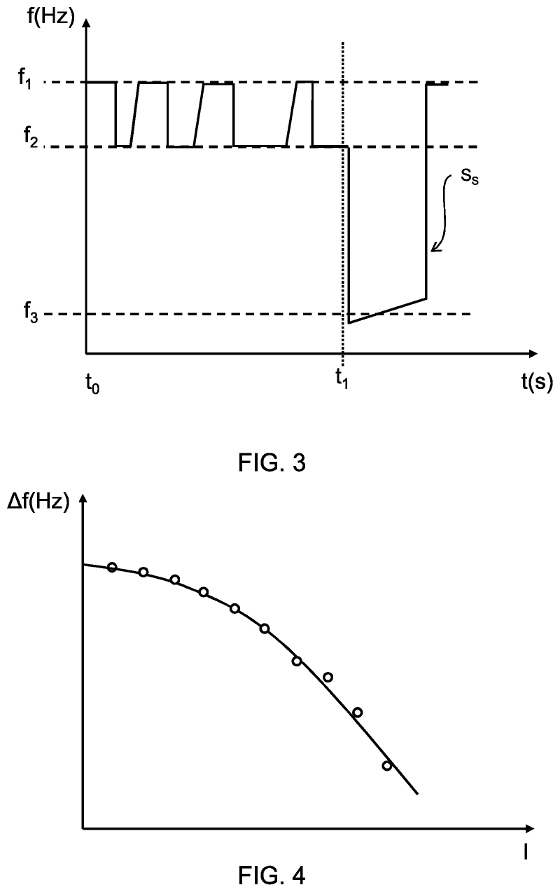

FIG. 3 illustrates the effect of ultrasound on a parameter of a sensing signal S_{S} generated by a capacitive pressure sensing arrangement according to previously described examples. Throughout the period illustrated by FIG. 3, the pressure applied to the capacitor by features other than the ultrasound wave(s) is constant.

The illustrated parameter 300 is here a measure of frequency of a sensing signal. The capacitive pressure sensing arrangement is configured to reduce a frequency of the sensing signal responsive to a capacitance of the capacitor of the capacitive pressure sensing arrangement increasing.

During a first period of time, from t₀ to t₁, ultrasound waves are alternately incident and not incident upon the capacitive pressure sensing arrangement. This demonstrates how when ultrasound waves are incident upon the capacitive pressure sensing arrangement, a parameter of the sensing signal changes (here: a frequency of the sensing signal drops). When no ultrasound waves are incident on the capacitive pressure sensing arrangement, the frequency of the sensing signal is at a first value f₁. When ultrasound waves are incident on the capacitive pressure sensing arrangement, the frequency of the sensing signal is at a second value f₂.

During a second period of time, from t₁ onwards, ultrasound waves are incident upon the capacitive pressure sensing arrangement, but have a greater power that those incident during the first time period t₀ to t₁. It can be seen that the greater power of the ultrasound waves during the second period of time increases the effect (i.e. the magnitude of the change) on the parameter of the sensing signal.

Thus, it will be understood that the difference between a first value f₁ (when no ultrasound waves are incident on the capacitive pressure sensing arrangement) and a second value f₂ (when ultrasound waves are incident on the capacitive pressure sensing arrangement) can be used to determine a presence of (and optionally the power of) the ultrasound wave at the position of the capacitive pressure sensing arrangement. This information can be used to track a position of the capacitive pressure sensing arrangement, and thereby the interventional device. For instance, the relationship between magnitude of an ultrasound wave and position of the capacitive pressure sensing arrangement can be known or the detected presence of the ultrasound wave can be used to triangulate the position of the interventional device.

FIG. 4 illustrates the relationship between magnitude of ultrasound wave I (received at the capacitor of the capacitive pressure sensing arrangement) and the magnitude of the parameter of the sensing signal to the ultrasound wave.

This Figure illustrates how, as the magnitude of ultrasound wave intensity I increases, so there is a greater magnitude of change in the parameter of the sensing signal, here the frequency of the sensing signal. Thus, the magnitude of the ultrasound wave received by the capacitive pressure sensing arrangement can be determined by processing a signal that changes in response to a change in pressure at a capacitor of the capacitive pressure sensing arrangement.

Thus, it can be understood how the presence and magnitude of an ultrasound wave can be identified by processing the native sensing signal produced by the capacitive pressure sensing arrangement, i.e. without needing further modification to the structure and/or configuration of the capacitive pressure sensing arrangement.

To discriminate between changes in the sensing signal resulting from an ultrasound wave, and changes in the sensing signal resulting from changes in one or more anatomical features of the patient, such as changes in blood pressure, the present disclosure proposes to make use of a predetermined modulation pattern.

In particular, the ultrasound waves may be emitted according to a predetermined modulation pattern. In some examples, the starting and stopping (i.e. the initiation and termination) transmission of ultrasound waves may follow a predetermined and identifiable modulation pattern. In another example, the magnitude of the emitted ultrasound waves follows a predetermined modulation pattern, e.g. alternating between a first (preferably non-zero) magnitude and a second (preferably non-zero) magnitude. Of course, more than two magnitudes could be used.

In the context of the present disclosure, a magnitude of an ultrasound wave refers to the intensity of the ultrasound wave when first emitted. This may correspond to a voltage provided to the transducer element(s) of the ultrasound transducer when emitting an ultrasound wave. This is to be distinguished from the magnitude of an ultrasound wave incident on the capacitive pressure sensing arrangement, which refers to the intensity of the ultrasound wave when it is incident upon the capacitive pressure sensing arrangement. This changes as the distance between the capacitive pressure sensing arrangement and the ultrasound transducer changes.

This predetermined pattern can be identified in the sensing signal generated by the capacitive pressure sensing arrangement, and used to identify a magnitude of the changes in the sensing signal resulting from the ultrasound wave. As previously explained, the response of a sensing signal to an ultrasound wave is dependent upon the intensity/magnitude of an ultrasound wave, so that a known variation or pattern in the intensity of ultrasound wave (e.g. between OFF and ON or between first and second non-zero values) can be identified in the sensing signal.

In this way, a first response of the sensing signal to the ultrasound waves can be extracted from the original sensing signal, by processing the sensing signal based on the modulation pattern.

This information can in turn be processed to track a location of the interventional device with respect to the ultrasound transducer.

For example, the information can be processed to determine a difference between a first value of a parameter of the sensing signal and a second value of the same parameter of the sensing signal. The first value may be obtained whilst the sensing signal is modified by an ultrasound wave (according to the identified modulation pattern) and the second value is obtained whilst the sensing signal is not modified by any ultrasound wave (according to the identified modulation pattern). The size of this difference will vary depending upon a distance between the ultrasound transducer and the capacitive pressure sensing arrangement, as the intensity of the ultrasound wave incident upon the capacitive pressure sensing arrangement varies according to distance. The determined difference can then be processed using a tracking algorithm to thereby track the position of the interventional device.

The parameter of the sensing signal is a parameter that is modified in response to a change in capacitance of the capacitor of the capacitive pressure sensing arrangement. Thus, in some examples, the parameter may be a frequency of an alternating signal generated by the capacitive pressure sensing arrangement.

In one example of a tracking algorithm, the difference of the parameter of the sensing signal may be processed to identify a magnitude of the ultrasound wave intensity received by the capacitive pressure sensing arrangement. It has been identified that there is a relationship between magnitude of the ultrasound wave intensity received by the capacitive pressure sensing arrangement and the change in capacitance of the capacitor of the capacitive pressure sensing arrangement (i.e. the perceived change in pressure). This was previously explained with reference to FIGS. 3 and 4. The magnitude of the ultrasound wave intensity can then be correlated to a position with respect to the ultrasound transducer—as the relationship between position and ultrasound wave intensity can be known or predetermined.

In particular, a response of the capacitive pressure sensing arrangement to an ultrasound wave indicates that the ultrasound wave has been emitted in a direction of the capacitive pressure sensing arrangement.

A position may be defined as a relative direction and distance from the ultrasound transducer to the capacitive pressure sensing arrangement.

As another example, a tracking algorithm may be performed through the use of multilateration techniques between the capacitive pressure sensing arrangement and at least two or three more points on the ultrasound transducer and/or two or more different positions of the ultrasound transducer.

During a conventional ultrasound imaging process, an emitted ultrasound beam is moved over a range of directions, i.e. over a range of angles, to generate the ultrasound image (from echoes to the beam over the range of angles). This facilitates identification of a direction or angle between the ultrasound transducer and the capacitive pressure sensing arrangement, as the capacitor will respond to a change in pressure induces by the ultrasound waves only when a direction of the ultrasound beam is in the direction of the capacitive pressure sensing arrangement.

Based on this understanding, it will be understood that determining a position of the capacitive pressure sensing arrangement may be performed by, when obtaining a first ultrasound image at a first position, determining a first angle/direction between the ultrasound probe and the capacitive pressure sensing arrangement and, when obtaining a second, later ultrasound image at a second (different) position, determining a second angle/direction between the ultrasound probe and the capacitive pressure sensing arrangement. A movement of the ultrasound probe between taking the first and second ultrasound images can also be tracked. This approach facilitates the triangulation of the distance (and hence position) of the capacitive pressure sensing arrangement, by identifying the position at which a first hypothetical line (having an origin at the first position and having a direction along the first direction) intersects a second hypothetical line (having an origin at the second position and having a direction along the second direction).

As another example, the ultrasound transducer may sequentially emit (or according to some other predetermined pattern) ultrasound waves from at least two different ultrasound elements of the ultrasound transducer (i.e. different areas on the ultrasound transducer), with the response of the capacitor to the ultrasound waves of different elements being used to effectively pinpoint/determine the position of the capacitor and/or capacitive pressure sensing arrangement with respect to the ultrasound transducer.

The transmission of the different ultrasound waves from different ultrasound transducers may follow a predetermined modulation pattern, which can be followed.

In another example, to determine distance between the ultrasound transducer and the capacitive pressure sensing arrangement, the focal length of the ultrasound transducer may be adjusted during an imaging process. In particular, the focal length of the ultrasound transducer may be adjusted whilst it is emitting a beam in a direction of the capacitive pressure sensing arrangement (as detected by the capacitor responding to the ultrasound beam). When the response of the capacitive pressure sensing arrangement reaches a maximum (i.e. there is a greatest detected amount of pressure change caused by the ultrasound wave(s)), this indicates that the focal length is approximately equal to the distance between the ultrasound transducer and the capacitive pressure sensing arrangement. Thus, a position of the capacitive pressure sensing arrangement with respect to the ultrasound transducer can be identified (as the angle and distance are known).

As yet another example, a time of flight measurement can be taken between a time at which the ultrasound beam is emitted at the time at which the capacitor of the capacitive pressure sensing arrangement responds to the ultrasound beam. The time of flight measurement may be correlated to a distance measurement (e.g. using a formula or lookup table), to predict a distance between the ultrasound transducer and the capacitive pressure sensing arrangement. This option is less preferred as the time resolution of the capacitive pressure sensing arrangement may be relatively poor.

Other suitable examples of a tracking algorithm will become apparent to the skilled person based on the teaching that the apparent capacitance of the capacitor of the capacitive pressure sensing arrangement differs based upon the relative position of the capacitive pressure sensing arrangement (and therefore the interventional device) with respect to an ultrasound transducer emitting ultrasound waves.

For instance, one example of a suitable tracking algorithm can be understood from European Patent Application No. EP 2446295 A1.

In some examples, to generate a measure of pressure resulting from one or more anatomical features of the patient, any identified predetermined modulation patterns may be taken into account. By way of example, a filter may be applied to offset changes caused by a predetermined modulation pattern. As another example, values of the sensing signal for generating the measure of pressure may be obtained only when it is known that the sensing signal is unaffected by an ultrasound wave (e.g. according to the modulation pattern).

Thus, the process of generating a measure of pressure may comprise processing the sensing signal based on the predetermined modulation pattern to determine a second response of the sensing signal, the second response being a response of the sensing signal to a change in a capacitance of the capacitive pressure sensing arrangement that does not result from the ultrasound waves emitted by the ultrasound transducer; and generate the measure of pressure based on the second response of the sensing signal.

The measure of pressure may be any suitable measure of pressure, such as a measure of blood pressure (e.g. systolic and/or diastolic blood pressure), a fraction flow reserve (FFR) which may be approximated as: FFR˜PD/PA, in which PD is the pressure distal to a particular region (e.g. a lesion) and PA is the pressure proximal to the particular region.

To improve the acceptance angle of the interventional device, it is preferred that relatively low ultrasound frequencies (e.g. no more than 2 MHZ, e.g. no more than 1 MHZ) are used. This results in a wavelength that is large with respect to the size of the capacitive pressure sensing arrangement, leading to a larger acceptance angle.

It was previously noted that the invention is particularly advantageous when the capacitive pressure sensing arrangement comprises two or more membranes that deform responsive to a pressure on the capacitive pressure sensing arrangement (and also vibrate in the presence of an ultrasound wave). This is because the boundary conditions of such capacitive pressure sensing arrangements make the membranes broad banded, therefore considerable deformation (and thereby perceived change in pressure) occurs even when the ultrasound waves are not a natural frequency of the membranes.

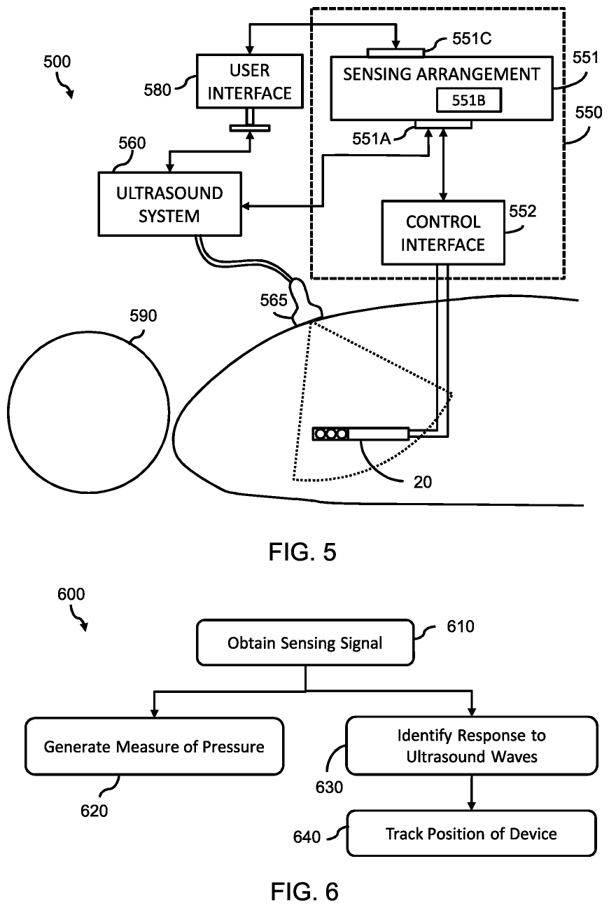

FIG. 5 illustrates an interventional medical system 500 according to an embodiment of the invention. The interventional medical system is designed for performing investigative operations on a patient 590.

The interventional medical system comprises an interventional device 20, such as those previously described.

The interventional medical system 500 further comprises a sensing system 550 for tracking a position of the interventional device within the patient 590. The sensing system comprises a sensing arrangement 551 and a control interface 552. The sensing system 550 and the sensing arrangement 551 both represent different embodiments of the invention.

The sensing arrangement 551 is configured to receive a sensing signal from the interventional device 20. As previously described, at least one parameter of the sensing signal is modified in response to a change in a capacitance on a capacitor the capacitive pressure sensing arrangement of the interventional device. For instance, the frequency of an alternating signal provided by the sensing signal may be responsive to a change in a capacitance of a capacitor of the capacitive pressure sensing arrangement that results from a change of pressure induced on the capacitor.

The sensing arrangement 551 receives this sensing signal via the control interface 552 of the sensing system 550.

The control interface 552 is configured to control all signals (including power and ground/reference voltages) provided to the capacitive pressure sensing arrangement of the interventional device and received from the capacitive pressure sensing arrangement of the interventional device. In particular, the control interface may provide/receive only 2 signals to the capacitive pressure sensing arrangement of the interventional device. That is, the control interface may connect to the interventional device using only two wires.

The control interface may provide a power signal (e.g. a voltage supply) and a reference voltage to the capacitive pressure sensing arrangement. The capacitive pressure sensing arrangement may modulate the power signal based on a capacitance of the capacitor of the capacitive pressure sensing arrangement.

The sensing arrangement 551 comprises an input interface 551A configured to obtain the sensing signal from the capacitive pressure sensing arrangement of the interventional device (here: via the control interface 552).

The sensing arrangement 551 further comprises a processing arrangement 551B configured to generate a measure of pressure by processing the sensing signal, wherein the measure of pressure is a measure of pressure resulting from one or more anatomical features of the patient.

The processing arrangement 551B is further configured to identify a first response of the sensing signal, the first response being a response of the sensing signal to the ultrasound waves emitted by the ultrasound transducer, by processing the sensing signal based on the predetermined modulation pattern.

The processing arrangement 551B is further configured to track the position of the interventional device with respect to the ultrasound transducer using the first response of the sensing signal.

Processes for tracking the position of the interventional device and for generate the measure of pressure have previously been described, and have been omitted for the sake of conciseness.

In some examples, the interventional medical system further comprises an ultrasound system 560 that controls the operation of an ultrasound transducer 565. To facilitate tracking of the interventional device 20 within the patient 590, the ultrasound system may modulate the emission of ultrasound waves by the ultrasound transducer according to a predetermined modulation pattern. This facilitates identification of the response of the capacitive pressure sensing arrangement to ultrasound within the sensing signal, as previously explained.

Moreover, the ultrasound system may separately control the operation of different elements of the ultrasound transducer, e.g. according to some predetermined pattern, to facilitate location identification of the capacitive pressure sensing arrangement (and therefore interventional device) using multilateration or triangulation techniques. For instance, to facilitate multilateration between the interventional device and elements of the ultrasound transducer, the ultrasound system may control different elements of the ultrasound transducer to emit a burst of ultrasound waves at different times. The response of the sensing signal to each burst of ultrasound waves can be used to effectively identify a distance between the interventional device and each transducer element. This information can be used to identify a location of the interventional device with respect to the ultrasound transducer.

The ultrasound system may communicate with the processing arrangement 551B of the sensing arrangement to allow the processing arrangement to identify a response of the sensing signal to ultrasound waves, e.g. by passing information on the predetermined pattern to the processing arrangement.

To improve the acceptance angle of the interventional device, it is preferred that relatively low ultrasound frequencies (e.g. no more than 2 MHz, e.g. no more than 1 MHz) are used. This results in a wavelength that is large with respect to the size of the capacitive pressure sensing arrangement, leading to a larger acceptance angle.

Thus, the ultrasound system may be configured to control the ultrasound transducer to (when emitting ultrasound waves) emit ultrasound waves having a relatively low ultrasound frequency, e.g. a frequency no greater than 2 MHz, e.g. no greater than 1 MHz.

Preferably, the ultrasound system emits ultrasound waves at the natural frequency of the membrane(s) of the capacitive pressure sensing arrangement, to increase a deformation of the membrane (due to harmonics) and further increasing the effect of the perceived change in pressure.

The proposed invention is particularly advantageous if the capacitive pressure sensing arrangement comprises more than one membrane responsive to a change in pressure. This is the boundary conditions of such capacitive pressure sensing arrangements make the membranes broad banded, therefore considerable deformation occurs even when the ultrasound waves are not a natural frequency of the membranes.

Of course, the ultrasound system 560 may also generate one or more ultrasound images using the ultrasound transducer 565, methods of which are well established in the art.

The sensing arrangement 551 may further comprise an output interface 551C configured to provide one or more output signals responsive to the tracked position of the interventional device and/or the measure of pressure. In some examples, each output signal is a display signal configured to control a display provided by a user interface 580 responsive to the tracked position of the interventional device and/or the measure of pressure.

Accordingly, the interventional medical system 500 may further comprise a user interface 580 configured to receive an output signal from the sensing arrangement 551 and provide a display responsive to the tracked position of the interventional device and/or the measure of pressure.

Of course, the user interface 580 may be configured to display one or more ultrasound images generated by the ultrasound system.

The tracked position of the interventional device may be displayed relative to the displayed one or more ultrasound images, e.g. an indicator overlaying a position of the interventional device with respect to the region represented by the ultrasound image. This is possible because the positional relationship between the interventional device and the ultrasound transducer is known, as is the position relationship between the region represented by the ultrasound image and the ultrasound transducer (i.e. they are registered together).

FIG. 6 illustrates a method 600 according to an embodiment of the invention. The method can be performed by the sensing arrangement.

The computer-implemented method is designed for tracking a position of an interventional device, within a patient, with respect to an ultrasound transducer that emits ultrasound waves according to a predetermined modulation pattern, wherein the interventional device comprises a capacitive pressure sensing arrangement.

The computer-implemented method 600 comprises a step 610 of obtaining a sensing signal from the capacitive pressure sensing arrangement of the interventional device, wherein the sensing signal is responsive to a change in a capacitance of a capacitor of the capacitive pressure sensing arrangement that results from a change of pressure induced on the capacitor.

The computer-implemented method 600 comprises a step 620 of generating a measure of pressure by processing the sensing signal, wherein the measure of pressure is a measure of pressure resulting from one or more anatomical features of the patient.

The computer-implemented method 600 comprises a step 630 of identifying a first response of the sensing signal, the first response being a response of the sensing signal to the ultrasound waves emitted by the ultrasound transducer, by processing the sensing signal based on the predetermined modulation pattern.

The computer-implemented method 600 comprises a step 640 of tracking the position of the interventional device with respect to the ultrasound transducer using the first response of the sensing signal.

Steps 620 and the combination of steps 630, 640 can be performed in parallel.

It will be understood that disclosed methods are preferably computer-implemented methods. As such, there is also proposed the concept of a computer program comprising computer program code for implementing any described method when said program is run on a processing system, such as a computer or a set of distributed processors.

Different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

The present disclosure proposes a computer program (product) comprising instructions which, when the program is executed by a computer or processing system, cause the computer or processing system to carry out (the steps of) any herein described method. The computer program (product) may be stored on a non-transitory computer readable medium.

Similarly, there is also proposed a computer-readable (storage) medium comprising instructions which, when executed by a computer or processing system, cause the computer or processing system to carry out (the steps of) any herein described method. There is also proposed computer-readable data carrier having stored thereon the computer program (product) previously described. There is also proposed a data carrier signal carrying the computer program (product) previously described.

The computer-readable program may execute entirely on a single computer/processor, partly on the computer/processor, as a stand-alone software package, partly on the computer/processor and partly on a remote computer or entirely on the remote computer or server (e.g. using a distributed processor processing system). In the latter scenario, the remote computer may be connected to the computer/processor through any type of network, including a local area network (LAN) or a wide area network (WAN), or the connection may be made to an external computer (for example, through the Internet using an Internet Service Provider).

The present invention uses an ultrasound system to generate ultrasound waves via an ultrasound transducer that are usable to identify a location of an interventional device (having a capacitive pressure sensing arrangement) with respect to the ultrasound transducer.

For the sake of completeness, an operation of an ultrasound system will be hereafter described for contextual understanding.

The system comprises an array transducer 74 which has a transducer array 76 for transmitting ultrasound waves and receiving echo information. The transducer array 76 may comprise CMUT transducers; piezoelectric transducers, formed of materials such as PZT or PVDF; or any other suitable transducer technology. In this example, the transducer array 76 is a two-dimensional array of transducers 78 capable of scanning either a 2D plane or a three dimensional volume of a region of interest. In another example, the transducer array may be a 1D array.

The transducer array 76 is coupled to a microbeamformer 712 which controls reception of signals by the transducer elements. Microbeamformers are capable of at least partial beamforming of the signals received by sub-arrays, generally referred to as “groups” or “patches”, of transducers as described in U.S. Pat. No. 5,997,479 (Savord et al.), U.S. Pat. No. 6,013,032 (Savord), and U.S. Pat. No. 6,623,432 (Powers et al.).

It should be noted that the microbeamformer is entirely optional. Further, the system includes a transmit/receive (T/R) switch 716, which the microbeamformer 712 can be coupled to and which switches the array between transmission and reception modes, and protects the main beamformer 720 from high energy transmit signals in the case where a microbeamformer is not used and the transducer array is operated directly by the main system beamformer. The transmission of ultrasound beams from the transducer array 76 is directed by a transducer controller 718 coupled to the microbeamformer by the T/R switch 716 and a main transmission beamformer (not shown), which can receive input from the user's operation of the user interface or control panel 738. The controller 718 can include transmission circuitry arranged to drive the transducer elements of the array 76 (either directly or via a microbeamformer) during the transmission mode.

In a typical line-by-line imaging sequence, the beamforming system within the transducer may operate as follows. During transmission, the beamformer (which may be the microbeamformer or the main system beamformer depending upon the implementation) activates the transducer array, or a sub-aperture of the transducer array. The sub-aperture may be a one dimensional line of transducers or a two dimensional patch of transducers within the larger array. In transmit mode, the focusing and steering of the ultrasound beam generated by the array, or a sub-aperture of the array, are controlled as described below.

Upon receiving the backscattered echo signals from the subject, the received signals undergo receive beamforming (as described below), in order to align the received signals, and, in the case where a sub-aperture is being used, the sub-aperture is then shifted, for example by one transducer element. The shifted sub-aperture is then activated and the process repeated until all of the transducer elements of the transducer array have been activated.

For each line (or sub-aperture), the total received signal, used to form an associated line of the final ultrasound image, will be a sum of the voltage signals measured by the transducer elements of the given sub-aperture during the receive period. The resulting line signals, following the beamforming process below, are typically referred to as radio frequency (RF) data. Each line signal (RF data set) generated by the various sub-apertures then undergoes additional processing to generate the lines of the final ultrasound image. The change in amplitude of the line signal with time will contribute to the change in brightness of the ultrasound image with depth, wherein a high amplitude peak will correspond to a bright pixel (or collection of pixels) in the final image. A peak appearing near the beginning of the line signal will represent an echo from a shallow structure, whereas peaks appearing progressively later in the line signal will represent echoes from structures at increasing depths within the subject.

One of the functions controlled by the transducer controller 718 is the direction in which beams are steered and focused. Beams may be steered straight ahead from (orthogonal to) the transducer array, or at different angles for a wider field of view. The steering and focusing of the transmit beam may be controlled as a function of transducer element actuation time.

Two methods can be distinguished in general ultrasound data acquisition: plane wave imaging and “beam steered” imaging. The two methods are distinguished by a presence of the beamforming in the transmission (“beam steered” imaging) and/or reception modes (plane wave imaging and “beam steered” imaging).

Looking first to the focusing function, by activating all of the transducer elements at the same time, the transducer array generates a plane wave that diverges as it travels through the subject. In this case, the beam of ultrasonic waves remains unfocused. By introducing a position dependent time delay to the activation of the transducers, it is possible to cause the wave front of the beam to converge at a desired point, referred to as the focal zone. The focal zone is defined as the point at which the lateral beam width is less than half the transmit beam width. In this way, the lateral resolution of the final ultrasound image is improved.

For example, if the time delay causes the transducer elements to activate in a series, beginning with the outermost elements and finishing at the central element(s) of the transducer array, a focal zone would be formed at a given distance away from the transducer, in line with the central element(s). The distance of the focal zone from the transducer will vary depending on the time delay between each subsequent round of transducer element activations. After the beam passes the focal zone, it will begin to diverge, forming the far field imaging region. It should be noted that for focal zones located close to the transducer array, the ultrasound beam will diverge quickly in the far field leading to beam width artifacts in the final image. Typically, the near field, located between the transducer array and the focal zone, shows little detail due to the large overlap in ultrasound beams. Thus, varying the location of the focal zone can lead to significant changes in the quality of the final image.

It should be noted that, in transmit mode, only one focus may be defined unless the ultrasound image is divided into multiple focal zones (each of which may have a different transmit focus).

In addition, upon receiving the echo signals from within the subject, it is possible to perform the inverse of the above described process in order to perform receive focusing. In other words, the incoming signals may be received by the transducer elements and subject to an electronic time delay before being passed into the system for signal processing. The simplest example of this is referred to as delay-and-sum beamforming. It is possible to dynamically adjust the receive focusing of the transducer array as a function of time.

Looking now to the function of beam steering, through the correct application of time delays to the transducer elements it is possible to impart a desired angle on the ultrasound beam as it leaves the transducer array. For example, by activating a transducer on a first side of the transducer array followed by the remaining transducers in a sequence ending at the opposite side of the array, the wave front of the beam will be angled toward the second side. The size of the steering angle relative to the normal of the transducer array is dependent on the size of the time delay between subsequent transducer element activations.

Further, it is possible to focus a steered beam, wherein the total time delay applied to each transducer element is a sum of both the focusing and steering time delays. In this case, the transducer array is referred to as a phased array.

In case of the CMUT transducers, which require a DC bias voltage for their activation, the transducer controller 718 can be coupled to control a DC bias control 745 for the transducer array. The DC bias control 745 sets DC bias voltage(s) that are applied to the CMUT transducer elements.

For each transducer element of the transducer array, analog ultrasound signals, typically referred to as channel data, enter the system by way of the reception channel. In the reception channel, partially beamformed signals are produced from the channel data by the microbeamformer 712 and are then passed to a main receive beamformer 720 where the partially beamformed signals from individual patches of transducers are combined into a fully beamformed signal, referred to as radio frequency (RF) data. The beamforming performed at each stage may be carried out as described above, or may include additional functions. For example, the main beamformer 720 may have 128 channels, each of which receives a partially beamformed signal from a patch of dozens or hundreds of transducer elements. In this way, the signals received by thousands of transducers of a transducer array can contribute efficiently to a single beamformed signal.

The beamformed reception signals are coupled to a signal processor 722. The signal processor 722 can process the received echo signals in various ways, such as: band-pass filtering; decimation; I and Q component separation; and harmonic signal separation, which acts to separate linear and nonlinear signals so as to enable the identification of nonlinear (higher harmonics of the fundamental frequency) echo signals returned from tissue and micro-bubbles. The signal processor may also perform additional signal enhancement such as speckle reduction, signal compounding, and noise elimination. The band-pass filter in the signal processor can be a tracking filter, with its pass band sliding from a higher frequency band to a lower frequency band as echo signals are received from increasing depths, thereby rejecting noise at higher frequencies from greater depths that is typically devoid of anatomical information.

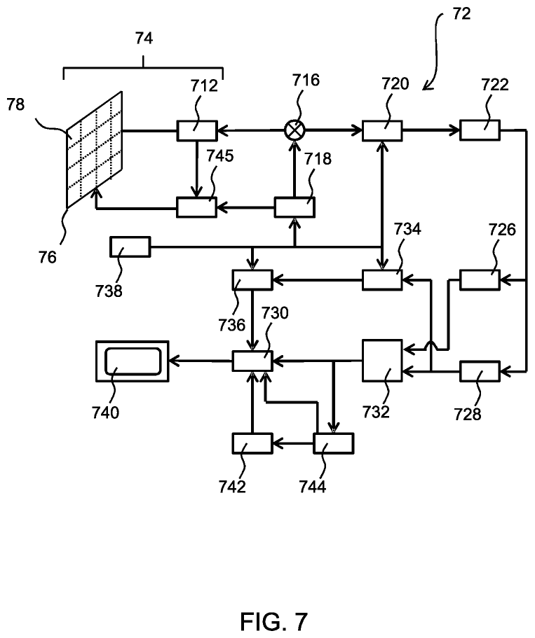

The beamformers for transmission and for reception are implemented in different hardware and can have different functions. Of course, the receiver beamformer is designed to take into account the characteristics of the transmission beamformer. In FIG. 7 only the receiver beamformers 712, 720 are shown, for simplicity. In the complete system, there will also be a transmission chain with a transmission micro beamformer, and a main transmission beamformer.

The function of the micro beamformer 712 is to provide an initial combination of signals in order to decrease the number of analog signal paths. This is typically performed in the analog domain.

The final beamforming is done in the main beamformer 720 and is typically after digitization.

The transmission and reception channels use the same transducer array 76 which has a fixed frequency band. However, the bandwidth that the transmission pulses occupy can vary depending on the transmission beamforming used. The reception channel can capture the whole transducer bandwidth (which is the classic approach) or, by using bandpass processing, it can extract only the bandwidth that contains the desired information (e.g. the harmonics of the main harmonic).

The RF signals may then be coupled to a B mode (i.e. brightness mode, or 2D imaging mode) processor 726 and a Doppler processor 728. The B mode processor 726 performs amplitude detection on the received ultrasound signal for the imaging of structures in the body, such as organ tissue and blood vessels. In the case of line-by-line imaging, each line (beam) is represented by an associated RF signal, the amplitude of which is used to generate a brightness value to be assigned to a pixel in the B mode image. The exact location of the pixel within the image is determined by the location of the associated amplitude measurement along the RF signal and the line (beam) number of the RF signal. B mode images of such structures may be formed in the harmonic or fundamental image mode, or a combination of both as described in U.S. Pat. No. 6,283,919 (Roundhill et al.) and U.S. Pat. No. 6,458,083 (Jago et al.) The Doppler processor 728 processes temporally distinct signals arising from tissue movement and blood flow for the detection of moving substances, such as the flow of blood cells in the image field. The Doppler processor 728 typically includes a wall filter with parameters set to pass or reject echoes returned from selected types of materials in the body.

The structural and motion signals produced by the B mode and Doppler processors are coupled to a scan converter 732 and a multi-planar reformatter 744. The scan converter 732 arranges the echo signals in the spatial relationship from which they were received in a desired image format. In other words, the scan converter acts to convert the RF data from a cylindrical coordinate system to a Cartesian coordinate system appropriate for displaying an ultrasound image on an image display 740 (which may be the user interface previously described). In the case of B mode imaging, the brightness of pixel at a given coordinate is proportional to the amplitude of the RF signal received from that location. For instance, the scan converter may arrange the echo signal into a two dimensional (2D) sector-shaped format, or a pyramidal three dimensional (3D) image. The scan converter can overlay a B mode structural image with colors corresponding to motion at points in the image field, where the Doppler-estimated velocities to produce a given color. The combined B mode structural image and color Doppler image depicts the motion of tissue and blood flow within the structural image field. The multi-planar reformatter will convert echoes that are received from points in a common plane in a volumetric region of the body into an ultrasound image of that plane, as described in U.S. Pat. No. 6,443,896 (Detmer). A volume renderer 742 converts the echo signals of a 3D data set into a projected 3D image as viewed from a given reference point as described in U.S. Pat. No. 6,530,885 (Entrekin et al.).

The 2D or 3D images are coupled from the scan converter 732, multi-planar reformatter 744, and volume renderer 742 to an image processor 730 for further enhancement, buffering and temporary storage for display on an image display 740. The imaging processor may be adapted to remove certain imaging artifacts from the final ultrasound image, such as: acoustic shadowing, for example caused by a strong attenuator or refraction; posterior enhancement, for example caused by a weak attenuator; reverberation artifacts, for example where highly reflective tissue interfaces are located in close proximity; and so on. In addition, the image processor may be adapted to handle certain speckle reduction functions, in order to improve the contrast of the final ultrasound image.

In addition to being used for imaging, the blood flow values produced by the Doppler processor 728 and tissue structure information produced by the B mode processor 726 are coupled to a quantification processor 734. The quantification processor produces measures of different flow conditions such as the volume rate of blood flow in addition to structural measurements such as the sizes of organs and gestational age. The quantification processor may receive input from the user control panel 738, such as the point in the anatomy of an image where a measurement is to be made.

Output data from the quantification processor is coupled to a graphics processor 736 for the reproduction of measurement graphics and values with the image on the display 740, and for audio output from the display device 740. The graphics processor 736 can also generate graphic overlays for display with the ultrasound images. These graphic overlays can contain standard identifying information such as patient name, date and time of the image, imaging parameters, and the like. For these purposes the graphics processor receives input from the user interface 738, such as patient name. The user interface is also coupled to the transmit controller 718 to control the generation of ultrasound signals from the transducer array 76 and hence the images produced by the transducer array and the ultrasound system. The transmit control function of the controller 718 is only one of the functions performed. The controller 718 also takes account of the mode of operation (given by the user) and the corresponding required transmitter configuration and band-pass configuration in the receiver analog to digital converter. The controller 718 can be a state machine with fixed states.

The user interface is also coupled to the multi-planar reformatter 744 for selection and control of the planes of multiple multi-planar reformatted (MPR) images which may be used to perform quantified measures in the image field of the MPR images.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims. A computer program may be stored/distributed on a suitable medium, such as an optical storage medium or a solid-state medium supplied together with or as part of other hardware, but may also be distributed in other forms, such as via the Internet or other wired or wireless telecommunication systems.

The mere fact that certain measures are recited in mutually different dependent claims does not indicate that a combination of these measures cannot be used to advantage. If the term “adapted to” is used in the claims or description, it is noted that the term “adapted to” is intended to be equivalent to the term “configured to”. Any reference signs in the claims should not be construed as limiting the scope.

1. A sensing arrangement for tracking a position of an interventional device, within a patient, with respect to an ultrasound transducer that emits ultrasound waves according to a predetermined modulation pattern, wherein the interventional device comprises a capacitive pressure sensing arrangement, wherein the sensing arrangement comprises:
an input interface configured to obtain a sensing signal from the capacitive pressure sensing arrangement of the interventional device, wherein the sensing signal is responsive to a change in a capacitance of a capacitor of the capacitive pressure sensing arrangement that results from a change of pressure induced on the capacitor;
a processing arrangement configured to:
generate a measure of pressure by processing the sensing signal, wherein the measure of pressure is a measure of pressure resulting from one or more anatomical features of the patient; and
identify a first response of the sensing signal, the first response being a response of the sensing signal to the ultrasound waves emitted by the ultrasound transducer, by processing the sensing signal based on the predetermined modulation pattern; and
track the position of the interventional device with respect to the ultrasound transducer using the first response of the sensing signal.

2. The sensing arrangement of claim 1, wherein the processing arrangement is configured to generate the measure of pressure by:
processing the sensing signal based on the predetermined modulation pattern to determine a second response of the sensing signal, the second response being a response of the sensing signal to a change in a capacitance of the capacitive pressure sensing arrangement that does not result from the ultrasound waves emitted by the ultrasound transducer; and
generating the measure of pressure based on the second response of the sensing signal.

3. The sensing arrangement of claim 1, wherein the processing arrangement is configured to track the position of the interventional device by:
determining a difference between a first value of a parameter of the sensing signal and a second value of the same parameter of the sensing signal, wherein the first value is obtained whilst the sensing signal is modified by an ultrasound wave and the second value is obtained whilst the sensing signal is not modified by any ultrasound wave; and
processing the determined difference using a tracking algorithm to thereby track the position of the interventional device.

4. The sensing arrangement of claim 1, wherein the measure of pressure is a measure of fractional flow reserve.

5. The sensing arrangement of claim 1, further comprising an output interface configured to provide one or more output signals responsive to the tracked position of the interventional device and/or the measure of pressure.

6. The sensing arrangement of claim 5, wherein each output signal is a display signal configured to control a display provided by a user interface responsive to the tracked position of the interventional device and/or the measure of pressure.

7. The sensing arrangement of claim 1, wherein the sensing signal provides an alternating signal, the frequency of the alternating signal being responsive to a change in a capacitance of a capacitor of the capacitive pressure sensing arrangement that results from a change of pressure induced on the capacitor.

8. A sensing system for tracking a position of an interventional device, within a patient, with respect to an ultrasound transducer that emits ultrasound waves, wherein the interventional device comprises a capacitive pressure sensing arrangement, the sensing system comprising:
the sensing arrangement of claim 1;
a control interface configured to control all signals provided to the capacitive pressure sensing arrangement and to receive all signals generated or modified by the capacitive pressure sensing arrangement, wherein the control interface is configured to provide and/or receive only two signals from or to the capacitive pressure sensing arrangement.

9. A interventional medical system comprising:
an interventional device comprising a capacitive pressure sensing arrangement having a capacitor configured to change capacitance responsive to a pressure induced thereon, wherein the interventional device is configured to generate the sensing signal responsive to a change in a capacitance of a capacitor of the capacitive pressure sensing arrangement that results from a change of pressure induced on the capacitor; and
the sensing arrangement of claim 1.

10. The interventional medical system of claim 9, wherein the interventional device comprises:
a first electrode, configured to receive a first signal; and
a second electrode; configured to receive a second signal,
wherein the capacitive pressure sensing arrangement is configured to modify the first and/or second signal responsive to a change in the capacitance of the capacitor.

11. The interventional medical system of claim 10, when dependent upon claim 8, wherein the control interface of the sensing system is configured to: provide, to the first electrode, a voltage for powering the capacitive pressure sensing arrangement; and provide, to the second electrode, a ground voltage.

12. The interventional medical system of claim 9, further comprising an ultrasound transducer configured to alternately start and stop emitting ultrasound waves according to a predetermined modulation pattern.

13. The interventional medical system of claim 12, further comprising an ultrasound system configured to control the operation of the ultrasound transducer.

14. A computer-implemented method for tracking a position of an interventional device, within a patient, with respect to an ultrasound transducer that emits ultrasound waves according to a predetermined modulation pattern, wherein the interventional device comprises a capacitive pressure sensing arrangement, wherein the computer-implemented method comprises:
obtaining a sensing signal from the capacitive pressure sensing arrangement of the interventional device, wherein the sensing signal is responsive to a change in a capacitance of a capacitor of the capacitive pressure sensing arrangement that results from a change of pressure induced on the capacitor;
generating a measure of pressure by processing the sensing signal, wherein the measure of pressure is a measure of pressure resulting from one or more anatomical features of the patient;
identifying a first response of the sensing signal, the first response being a response of the sensing signal to the ultrasound waves emitted by the ultrasound transducer, by processing the sensing signal based on the predetermined modulation pattern; and
tracking the position of the interventional device with respect to the ultrasound transducer using the first response of the sensing signal.

15. A computer program product comprising instructions which, when executed by a suitable computer or processing system, cause the computer to carry out the method herein described.
